(19) United States
(12) Patent Application Publication
(10) Pub. No.: US 2008/0103550 A1
(43) Pub. Date: May 1, 2008

(54) MULTIPLE ELECTRODE WOUND HEALING PATCH

(76) Inventors: Stuart Wenzel, San Carlos, CA (US); Mariam Maghribi, Fremont, CA (US); Mark Huang, Pleasanton, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/876,147

(22) Filed: Oct. 22, 2007

Related U.S. Application Data
(60) Provisional application No. 60/863,417, filed on Oct. 30, 2006.

(51) Int. Cl. A61N 1/02

(52) U.S. Cl. 607/50

(57) ABSTRACT

In one example, the present invention is directed to a wound-healing patch including a flexible substrate, at least one wound electrode and at least one return electrode. In the invention, the wound electrode(s) is positioned on a portion of the flexible substrate designed to be placed over wounded tissue and the return electrode is positioned on a portion of the substrate remote from the wound-healing electrode and designed to be placed over healthy tissue.

### CROSS-REFERENCE

This application claims priority from Provisional Application No. 60/863,417 filed Oct. 30, 2006, entitled Electrodes and Electronics for Electrostimulated Wound-Healing Devices which application is fully incorporated herein by reference.

The present invention is directed to a wound healing patch and, more particularly, to an improved wound healing patch using multiple electrodes, including wound healing and return electrodes.

### BACKGROUND OF THE INVENTION

Wounds and their complications are a major problem in both hospital and home settings. Healing such wounds is a priority for those who work in the health care field. There are many types of wounds that have different associated complications. For example, diabetic ulcers are caused and exacerbated by poor blood flow and inflammation, and are slow to heal, or may never heal if left untreated. This can lead to infection and scarring, among other problems. Thus, devices that promote wound healing are highly beneficial. While band aids and other wound dressings assist in the healing process by protecting the wound and helping to absorb fluids, it would be beneficial to have a wound healing patch which actively promotes the healing process.

### SUMMARY OF THE INVENTION

In one embodiment, the present invention is directed to a wound-healing patch including a flexible substrate, at least one wound electrode and at least one return electrode. In this embodiment of the invention, the wound electrode(s) is positioned on a portion of the flexible substrate designed to be placed over wounded tissue. Further, in this embodiment of the invention, the return electrode(s) is positioned on a portion of the substrate remote from the wound-healing electrode and designed to be placed over healthy tissue.

In a further embodiment of the present invention, the wound-healing patch includes a voltage source connected between the wound electrode(s) and the return electrode(s). In a further embodiment of the present invention the wound-healing patch includes a current source connected between the wound electrode(s) and the return electrodes(s). In a further embodiment of the present invention, the wound-healing patch includes a resistor connected to the wound electrode(s) to control the flow of current into the wound. In a further embodiment of the present invention the wound-healing patch includes control electronics adapted to control the flow of current through the at least one wound electrode.

### BRIEF DESCRIPTION OF THE FIGURES

The following detailed description should be read with reference to the drawings, in which like elements in different drawings are identically numbered. The drawings, which are not necessarily to scale, depict selected exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient”, “host” and “subject” refer to any human or animal subject and are not intended to limit the systems or methods to human use, although use of the subject invention in a human patient represents a preferred embodiment.

The invention will now be described, by way of example only, with reference to the following figures. The accompanying drawings, which are incorporated herein and constitute part of this specification, illustrate presently preferred embodiments of the invention, and, together with the general description given above and the detailed description given below, serve to explain features of the invention, in which:

FIG. 1 illustrates an embodiment of a wound healing patch according to the present invention in which a return electrode is positioned far away from wound electrodes.

FIG. 2 illustrates the embodiment of a wound-healing patch according to the present invention illustrated in FIG. 1 wrapped around an arm.

FIG. 3 illustrates a further embodiment of a wound healing patch according to the present invention including distributed resistive electrodes formed using conductive layers on top of resistive layers.

FIG. 4 is a bottom view of a wound healing patch according to the present invention.

FIG. 5 illustrates a wound healing patch according to an embodiment of the present invention including distributed resistive electrodes and a voltage source.

FIG. 6 is diagram of an electrical model of a resistive layer useful in the present invention.

FIG. 7 illustrates a wound healing patch according to an embodiment of the present invention including distributed resistive electrodes and a contiguous resistive layer.

FIG. 8 illustrates a wound healing patch according to an embodiment of the present invention including a continuous resistive layer, multiple conductive wound electrodes and multiple conductive return electrodes.

FIG. 9 illustrates a wound healing patch according to an embodiment of the present invention including isolated distributed resistive electrodes and multiple conductive wound and return electrodes.

FIG. 10 illustrates a wound healing patch according to an embodiment of the present invention that includes isolated distributed resistive electrodes and multiple conductive wound and return electrodes where the return electrodes are in direct contact with tissue.

FIG. 11 illustrates a wound healing patch according to an embodiment of the present invention including a continuous resistive layer, and multiple conductive wound and return electrodes where the continuous resistive layer forms a structural layer.

FIG. 12A is a diagram of a wound covered by a wound healing patch according to the present invention with a circuit diagram illustrating resistance in the wound superimposed.

FIG. 12B schematically illustrates resistances and current flow in a wound-healing patch and wound.

### DETAILED DESCRIPTION OF THE FIGURES

The following detailed description should be read with reference to the drawings, in which like elements in different drawings are identically numbered. The drawings, which are not necessarily to scale, depict selected exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient”, “host” and “subject” refer to any human or animal subject and are not intended to limit the systems or methods to human use, although use of the subject invention in a human patient represents a preferred embodiment.

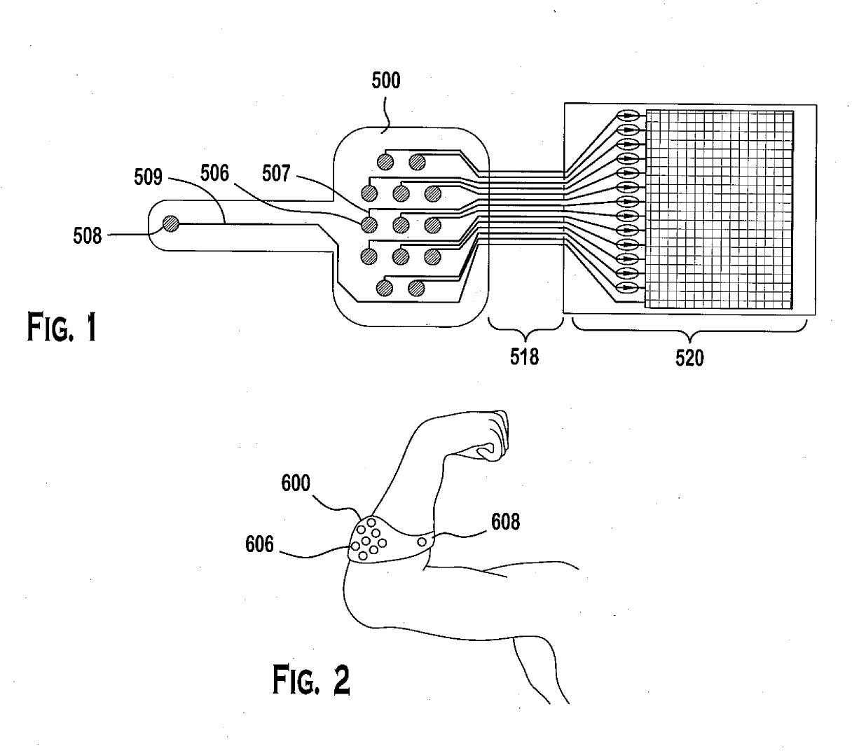

FIG. 1 illustrates an embodiment of the present invention in which a return electrode is positioned farther away from the wound electrodes. In the embodiment of the invention illustrated in FIG. 1, return electrode 508 is separated by a large distance from an array of wound electrodes 506. In using this embodiment, patch 500 may be wrapped around a limb, positioning return electrode 508 opposite wound electrodes 506. Since the most direct path for current flow is through limb tissue, current enters wound electrodes 506 by flowing substantially directly through the limb, improving current uniformity and reducing the need for a guard electrode that shunts lateral current. In another embodiment, this configuration may also include guard electrode or electrodes in order to further improve wound-current uniformity and to enable higher currents to be used without creating hot spots of high current near the periphery of the wound electrodes. The embodiment of the invention illustrated in FIG. 1 includes wound electrode traces 507, return electrode trace 509, connector cable 518, and external electronics package 520. External electronics package 520 may include a battery, a microprocessor, and/or a multiplexer. FIG. 2 illustrates an embodiment such as that illustrated in FIG. 1 in use. Patch 600 includes return electrode 608 and wound electrodes 606, and is wrapped around an arm. Current travels from return electrode 608, through the arm tissue, and into wound electrodes 606, substantially vertically and in that way reducing the need for a guard electrode.

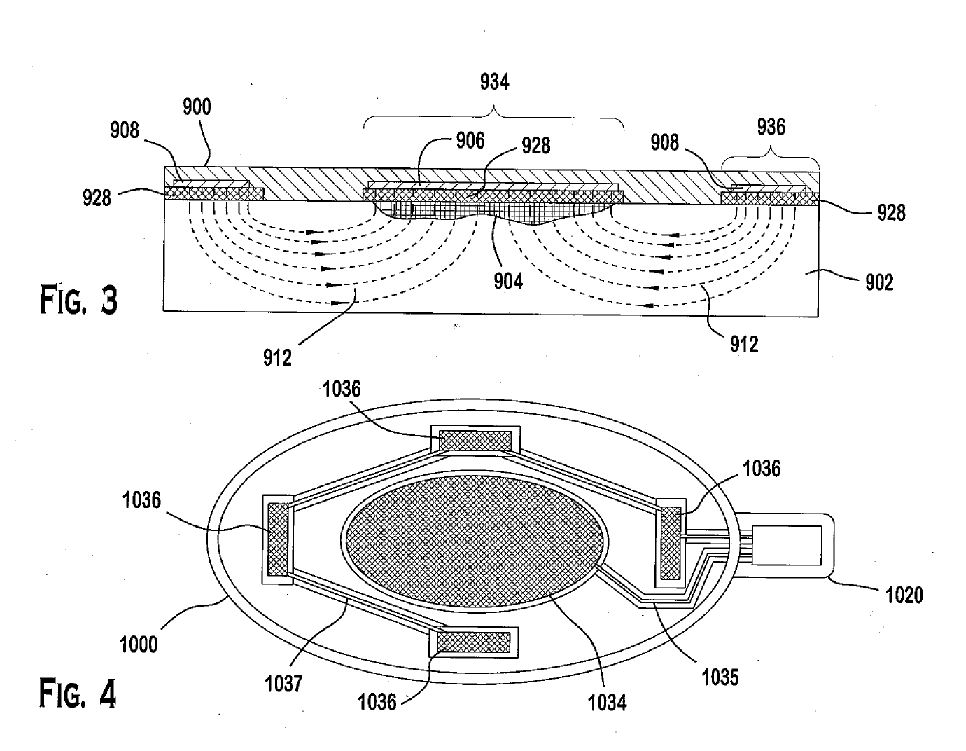

FIG. 3 illustrates a another embodiment of the present invention, wherein distributed resistive wound electrode 934 and distributed resistive return electrodes 936 are formed using wound electrode 906 and return electrodes 908 on top of resistive layers 928. In this embodiment, distributed resistive wound electrode 934 and distributed resistive return electrodes 936 deliver substantially uniform wound current 912 across wound 904. Distributed resistive wound electrode 934 is positioned directly over wound 904, and delivers uniform wound current 912. Resistive layers 928 provide cost effective means to deliver uniform current and electric field across wound 904. Resistive layers 928 can be formulated to provide various degrees of resistance, and can help prevent hot spots caused by high current flow. In some embodiments, resistive layers 928 eliminate the need for additional balancing resistors, and/or more costly independent current source electronics. Resistive layers 928 can be fabricated using various materials, such as carbon filled polymers, and/or polymers containing silver (which is antimicrobial). Resistive layers 928 can be screen printed, or can be laminated (using conductive adhesive bonds between layers). In designs where wound electrode 906 is much larger than wound 904, guard electrodes are not necessary, as portions of wound electrode 906 and resistive layers 928 can perform the same function as a guard electrode, preventing current from concentrating at the edge of wound 904. In general, resistive layers 928 prevent high current flow at any one particular spot, helping to distribute current uniformly across wound 904 and tissue 902.

FIG. 4 illustrates an embodiment of the invention utilizing a distributed resistive wound electrode 1034 and distributed resistive return electrodes 1036. Distributed resistive wound electrode traces 1035 and distributed resistive return electrode traces 1037 connect distributed resistive wound electrode 1034 and distributed resistive return electrodes 1036 to external electronics package 1020, and include an electrically insulating layer on top to prevent current leakage. External electronics package 1020 can include a battery, active and passive components, displays and wireless transmission components.

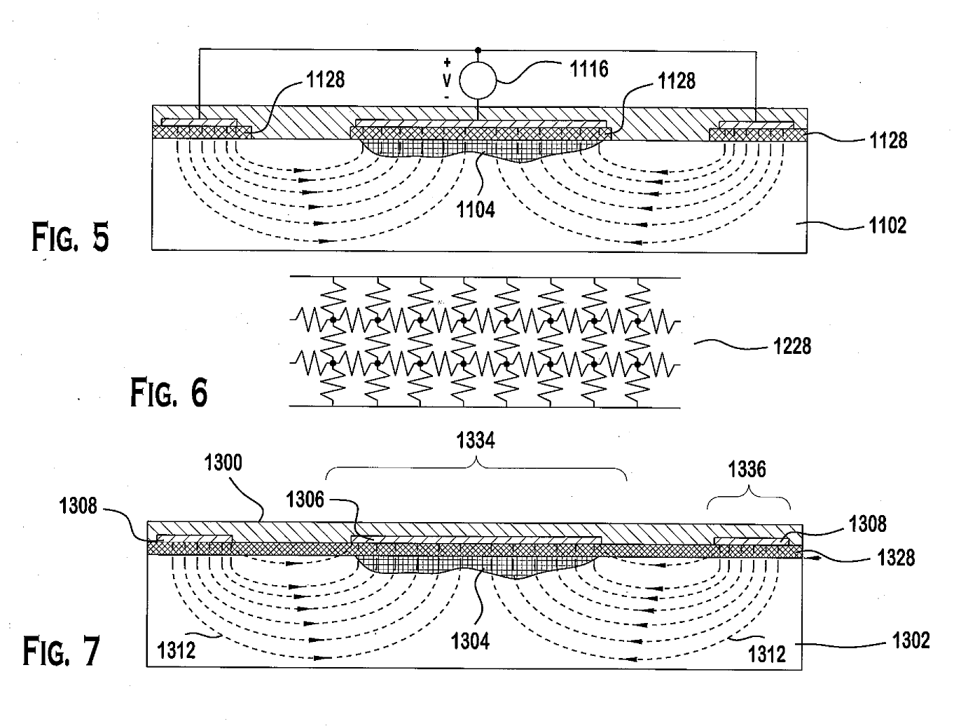

FIG. 5 illustrates an embodiment of the present invention including voltage source 1116. Voltage source 1116 can be used with distributed resistive electrode designs, such as that illustrated in FIG. 3. Voltage sources 1116 include all types of AC voltage sources, in addition to DC. In FIG. 5, resistive layer 1128 limits current, and is a low cost means to provide uniform current flow in tissue 1102 and wound 1104.

FIG. 6 is an electrical model of resistive layer 1228, useful in embodiments of the present invention such as that illustrated in FIG. 3. As illustrated in FIG. 6, resistive layer 1228 is essentially a uniform resistive network, and acts as a distributed resistor. The resistance through the thickness of resistive layer 1228 will generally be much higher than the tissue resistance between return and wound electrodes. This ensures that resistive layer 1228 controls and limits current, helping to provide uniform current density (Amps/cm²) throughout the wound area. The electrical resistivity of resistive layer 1228 will be in the range of 10−3-10⁺⁹ ohm-cm. (For reference, metallic conductors have much lower resistivity, e.g., copper resistivity is about 2×10⁻⁶ ohm-cm.) Resistive layer 1228 will generally be thin, on the order of 0.001-5 mm. Components that are electrically connected to resistive layer 1228 but are separated by a large distance compared to the thickness of resistive layer 1228 will have a large resistance between them, and are effectively electrically isolated. Therefore, it's possible to make devices that use resistive layer 1228 for structural purposes, eliminating the need for a top layer, as illustrated in FIG. 11.

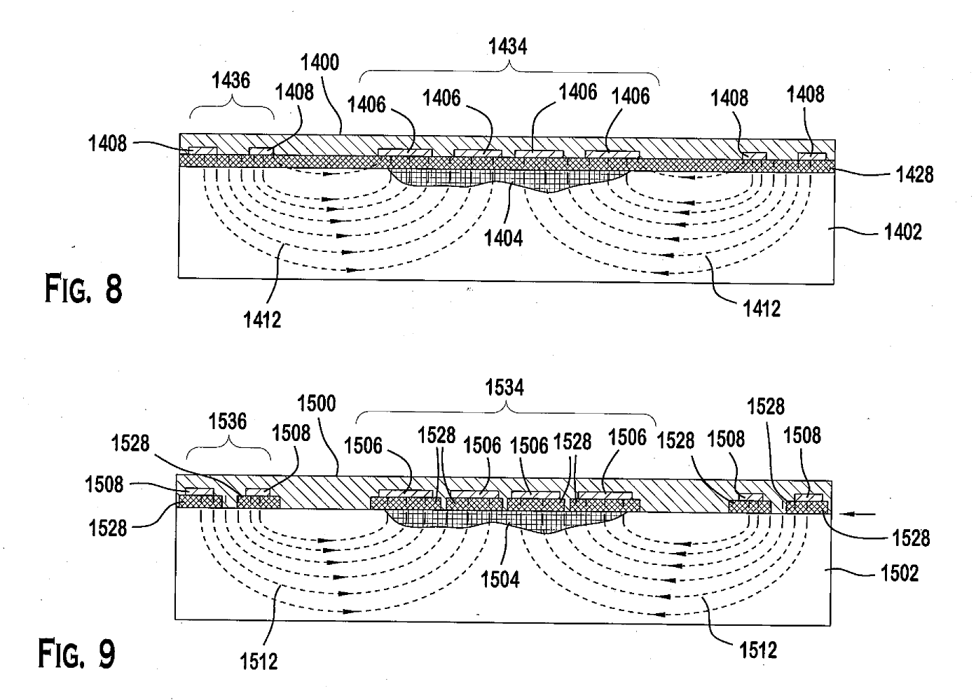

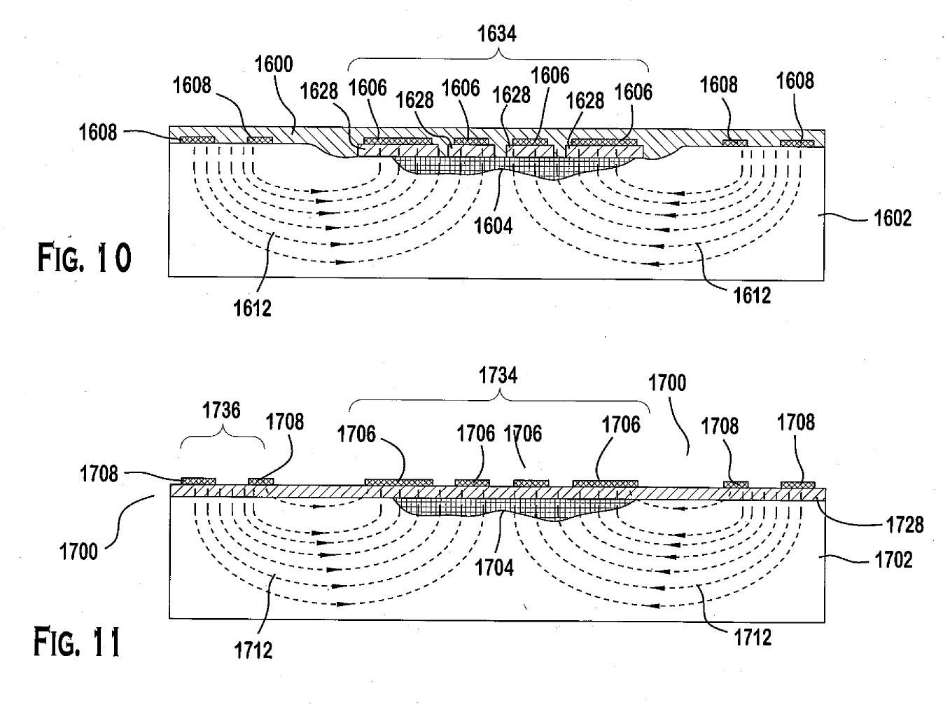

FIG. 7 illustrates an alternate embodiment of the present invention. Patch 1300 uses a contiguous resistive layer 1328. Patch 1300 includes distributed resistive wound electrode 1334 and distributed resistive return electrode 1336. Distributed resistive wound electrode 1334 includes wound electrode 1306 and contiguous resistive layer 1328, while distributed resistive return electrode 1336 include return electrodes 1308 and contiguous resistive layer 1328. When patch 1300 is placed in direct contact with tissue 1302 and wound 1304, wound current 1312 passes through tissue 1302 and wound 1304. In resistive layer 1328, resistance in the lateral direction is much higher than the resistance in tissue 1302. Typically, resistive layer 1328 will be thin enough compared to the width, which is obvious, that the vertical resistance will be lower than lateral, so that current takes the vertical path. We can also use z-axis materials, which allow current to flow only vertically and not laterally/horizontally. For this reason, resistive layer 1328 does not need to be printed in a pattern. Instead, a uniform resistive layer can be used. Resistive layer 1328 can be thick enough to provide structural integrity, as illustrated in the embodiment illustrated in FIG. 11. FIG. 8 illustrates another embodiment of the present invention. Patch 1400 includes distributed resistive wound electrode 1434, distributed resistive return electrode 1436, as well as contiguous resistive layer 1428. Distributed resistive wound electrode 1434 includes multiple wound electrodes 1406 and contiguous resistive layer 1428, while distributed resistive return electrode 1436 includes multiple return electrodes 1408 and contiguous resistive layer 1428. When patch 1400 is placed in direct contact with tissue 1402 and wound 1404, wound current 1412 passes through tissue 1402 and wound 1404. FIG. 9 illustrates an alternative embodiment of the present invention. Patch 1500 includes distributed resistive wound electrode 1534, distributed resistive return electrode 1536, as well multiple resistive layers 1528. Distributed resistive wound electrode 1534 includes multiple wound electrodes 1506 and multiple resistive layers 1528, while distributed resistive return electrode 1536 includes multiple return electrodes 1508 and multiple resistive layers 1528. Patch 1500 also includes multiple resistive layers 1528, multiple and multiple resistive layers 1528. When patch 1500 is placed in direct contact with tissue 1502 and wound 1504, wound current 1512 passes through tissue 1502 and wound 1504. Using discrete resistive layers 1528 allows discrete resistance values, if desired. Different resistive values may be useful in balancing current flow through tissue 1502 and wound 1504. FIG. 10 illustrates another embodiment of the present invention. As illustrated in FIG. 10, there is no resistive layer between return electrodes 1608 and tissue 1602. Patch 1600 includes distributed resistive wound electrode 1634, which includes wound electrodes 1606 and resistive layers 1628. When patch 1600 is placed in direct contact with tissue 1602 and wound 1604, wound current 1612 passes through tissue 1602 and wound 1604. The embodiment illustrated in FIG. 10 shows that in some embodiments of the present invention resistive layers are used only where necessary, in this case over wound electrodes 1606, but not over return electrodes 1608. FIG. 11 illustrates an alternative embodiment of the present invention. Patch 1700 includes distributed resistive wound electrode 1734, distributed resistive return electrode 1736, and contiguous resistive layer 1728. In this embodiment, resistive layer 1728 can be used as a structural layer as well (alleviating the need for a top polymer layer). Distributed resistive wound electrode 1734 includes multiple wound electrodes 1706 and resistive layer 1728, while distributed resistive return electrode 1736 includes multiple return electrodes 1708 and resistive layer 1728. When patch 1700 is placed in direct contact with tissue 1702 and wound 1704, wound current 1712 passes through tissue 1702 and wound 1704. Embodiments such as that illustrated in FIG. 11, which uses a single resistive layer 1728 for both electrical and structural reasons, significantly lowers the cost to manufacture patch 1700.

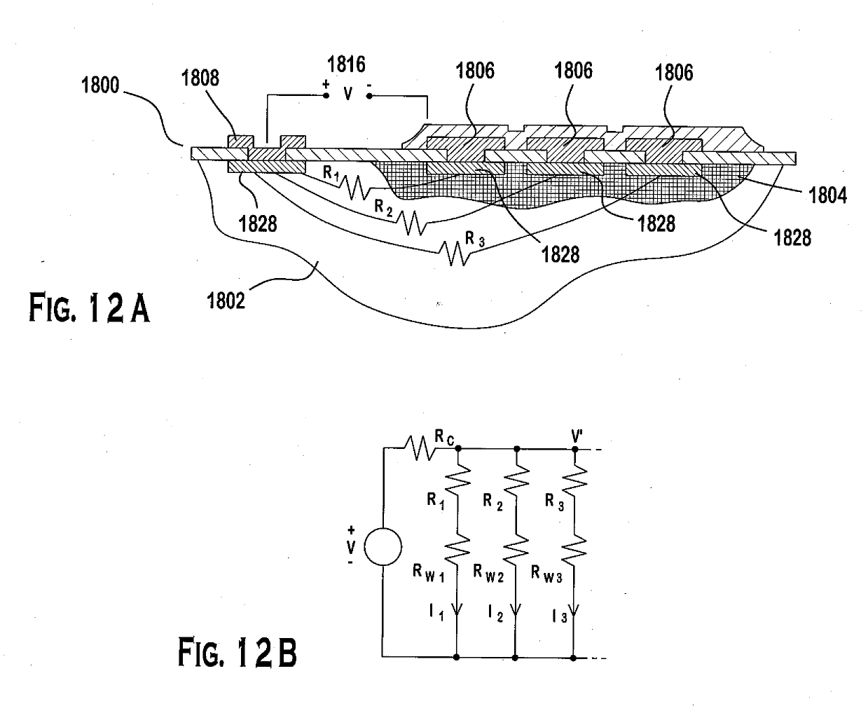

FIGS. 12A and 12B schematically illustrate resistances and current flow in a wound-healing patch 1800, according to an embodiment of the present invention. Patch 1800 includes wound electrodes 1,806, return electrode 1808, resistive layers 1828, and voltage source 1816. Voltage source 1816 can provide a variety of voltage signals, including DC, AC, pulsed, bi-polar, unipolar, or bursted. Wound-healing patch 1800 is mounted on tissue 1802, and is in contact with wound 1804. R_{w1}, R_{w2}, and R_{w3}, represent the electrical resistance of tissue 1802, and vary depending upon path length and the physical constituents of tissue 1802 (such as wound tissue, skin, muscle, bone, and fat). The resistances of wound electrodes 1806 and resistive layers 1828 combine to form resistances R₁, R₂, and R₃. The other resistances in the circuit around the voltage source are rolled up into resistor R_{c}; these resistances include the internal resistance of the voltage source and contact resistances. FIG. 12B is a schematic circuit diagram of patch 1800, in contact with tissue 1802 and wound 1804. I₁, I₂, and I₃ are wound current flows through the first, second, and third wound electrodes 1806. The magnitude of I₁, I₂, and I₃ can be calculated using equations (1) through (7). Determining the magnitude of wound currents I₁, I₂, and I₃ is useful in selecting the resistance values of resistive layers 1828, in sizing voltage source 1816, and in designing circuitry to balance current flow through tissue 1802 and wound 1804. Equation (1) can be used to calculate I₁, using known values of V, the voltage at voltage source 1816, along with R_{c}, R₁, and R_{w1}, Iₜₒₜ, the total current flow between return electrode 1808 and wound electrodes 1806, can be calculated using Equation 3, after calculating values for I₂ and I₃ (using Equation 2).

$$\begin{matrix}{I}_{1} = \frac{V - {I}_{tot} {R}_{c}}{{R}_{1} + {R}_{w 1}} & ( 1 ) \\ {I}_{i} = \frac{V - {I}_{tot} {R}_{c}}{{R}_{i} + {R}_{wi}} & ( 2 ) \\ {I}_{total} = \sum {I}_{1} + {I}_{2} + … = \sum {I}_{i} & ( 3 )\end{matrix}$$

In cases where the combined resistance of wound electrode 1806 and resistive layer 1828 is much greater than Rᵢ, the resistance of tissue 1804, Equation 2 can be simplified using the expressions shown in Equations 5-7. As expressed in Equation 7, wound current Iᵢ is directly proportional to V′, the voltage inside tissue 1802, and is inversely proportional to the combined resistance of wound electrode 1806 and resistive layer 1828. As mentioned previously, Equations 1-7 are useful in designing patch 1800, and assuring uniform current distribution through tissue 1802 and wound 1804.

$$\begin{matrix}{V}^{′} = V - {I}_{tot} {R}_{c} & ( 5 ) \\ {I}_{1} = \frac{{V}^{′}}{{R}_{1} + {R}_{w 1}} & ( 6 ) \\ {I}_{i} = \frac{{V}^{′}}{{R}_{i} + {R}_{wi}} \approx \frac{{V}^{′}}{{R}_{i}} for Ri >> {R}_{wi} . & ( 7 )\end{matrix}$$

### These calculations show that the resistance of the resistive layers 1828 can be chosen to be high (Rᵢ>>R_{wi}) such that approximately equal current Iᵢ passes through each wound electrode 1828 and 1806.

In a further embodiment of the present invention, alternating current or voltage may be used to force electrical current through the wound. In this embodiment, one or more wound electrode(s) is positioned over the wound, but no DC return electrode is provided. In this embodiment of the invention, electrical power, such as a voltage or current drive, would force AC or transient charge in and out of the wound through the wound electrode(s). A capacitor or super capacitor could be used to store accumulated charge. In some designs, an electrically isolated return electrode could be used to capacitively couple the charge in and out of the wound, but no net charge transfer would occur.

In some embodiments of the present invention, the signal can be varied over each part of the wound, to optimize wound healing.

In other embodiments of the present invention, measuring the electrical characteristics of the wound can assess the efficacy and rate of wound healing. Measurement circuitry can be connected to wound, return, or guard electrodes, and can measure physical parameters, such as temperature. A variety of circuitry can be used to measure temperature, including thermocouples and RTDs (resistance temperature device). RTDs can be made from resistive traces in the patch that change resistivity with temperature. Measurement of voltages, currents and electrical fields (voltage gradient), along with temperature inside and outside the wound, provides useful information for the patient and/or the health care provider.

In further embodiments of the present invention, impedance ratios may be measured using a wound-healing patch. The impedance is the ratio of applied voltage to current, each of which may be time varying. The impedance is a complex quantity (has real and imaginary components, or equivalently, magnitude and phase) and depends on frequency. Impedance parameters of the wound can be measured with electrodes and the proper electronics (on or off the patch). Two-wire or 4-wire configurations can be used to measure resistance and impedance. Impedance can be measured between any independent electrodes, including the return and wound electrodes.

In further embodiments of the present invention, wound-healing devices can also include different types of sensors to assess wound healing and the potential of infection. Sensors may measure chemicals, such as oxygen or VOCs (volatile organic compounds) that are exuded by infected wounds. Sensors could also measure the pH of the wound, or the wounds optical properties, such as reflection, and/or absorption and emission of light in the microwave, visible, infrared, or ultraviolet spectrums.

In further embodiments of the present invention, wound-healing assessment information can be relayed to the user or doctor with an electronic display, onboard or external to the patch. This might be a digital display (e.g., the LCD of a PDA) on the patch or external to it, or indicators on the patch such as LEDs (or organic LEDs).

In further embodiments of the present invention, wireless methods may be used to transmit data and power. A patch may include one or more antennas for this purpose, including coil antennas for inductive coupling, dipole antennas, phased arrays etc. Power can be coupled into the wound-healing patch inductively, for example. This scheme would permit a reliable, robust, waterproof power connector to be made with coil antennas that are fully encased in plastic, for example.

In further embodiments of the present invention, wireless telemetry can be used to transmit data into and from the wound-healing patch. Data from the patch can be transmitted to a receiver that displays data or relays it to a health-care professional who can make recommendations that are then transmitted back to the patient or directly to the wound-healing device to modify its operational parameters. Methods of telemetry that can be used include short-distance methods (such as inductive coupling and impedance modulation) and longer-distance transmission protocols such as AM, FM, cellphone, GSM, TDMA, 1XRTT, CDMA, EDGE, MICS, Bluetooth, Zigbee, 802.11a/b/g.

In further embodiments of the present invention, electronic functions can be housed on the patch or off the patch. This may include one or more ASICs. In other embodiments of the present invention, electronic signal can be made to vary over the wound area. That is, non-uniform current or voltage can be generated over the wound rather than uniform current density. In other embodiments of the present invention, feedback can be used to adjust the electric signal as the wound healing progresses. E.g., the current can be reduced in areas where healing is more complete. The signal can also be adjusted depending on the phase of healing (inflammation, proliferation, reconstruction etc). Completely different signals and polarity may be appropriate for the different phases. In other embodiments of the present invention, the short-term temporal profile of the voltage or current drive can be DC or AC, including sinusoidal, square wave, pulsed (<50% duty cycle), triangular, sawtooth, or tone burst profiles.

In further embodiments of the present invention, data can be displayed to the user and communicated to and from health-care professionals as part of the monitoring and control process. Communication might be via lights or displays mounted on a patch, or through wired or wireless transmission to/from nearby or distant locations. For example, in one embodiment of the present invention, simple data might only trigger a light on the patch that alerts the user to change the wound-healing patch. In another embodiment, data could be transmitted to a computer in the doctor's office that performs an analysis and warns of an infection in real time or with a short time delay. Self-test and calibration can be integrated into the system and performed upon initial application of the patch and at periodic intervals.

The present invention is particularly beneficial because multiple-electrode designs enable controlled delivery and measurement of electrical signals at each part of a wound. In a wound-healing patch according to the present invention, it is possible to apply equal or varied current density through all parts of a wound. Further, utilizing a patch according to the present invention, it is possible to ensure that current travels from deep tissue through the wound to the surface, substantially perpendicular to the surface, thus facilitating interaction with the deep healthy tissue and blood supply. Further, utilizing a wound-healing patch according to an embodiment of the present invention, it is possible to measure electrical and other wound parameters to assess healing. Further, in another embodiment of the present invention, it is possible to tailor the electrical signal (current or voltage) applied to each part of a wound to optimize local healing.

In any of these dressing designs, it may be advantageous to have the collective area of the wound electrodes smaller than that of the return electrodes. This causes the current density (A/cm²) and electric field to be highest in the tissue near the wound electrodes (i.e., in the wound itself). Another way to say this is that most of the applied voltage will appear across the smaller electrodes, i.e., between the wound electrodes and the tissue, rather than between the return electrodes and the tissue. This result follows directly from Ohm's law, which states that current density is proportional to electric field: E=ρJ, where E is the electric field (V/cm) and J is the current density (A/cm²) and ρ (ohm-cm) is the electrical resistivity of the material (the inverse of conductivity). This configuration could be used to maximize the efficacy of wound-healing while minimizing the energy drain of a battery or other power source.

The present invention is directed to wound-healing patches (bandages) with integrated electrodes, electronics and electrostimulation. Embodiments of the present invention employ multiple, independent electrodes covering the wound, wherein the electrodes can be used to deliver electrical signals, and can be used to measure electrical wound parameters. “Independent electrode” means an electrode that can be controlled separately from surrounding electrodes, including the ability to have a different electrical voltage or current than nearby electrodes. Control may be local and simple, as in a series resistor that limits electrode current while many electrodes are connected to the same voltage source (FIG. 2). Control may also be remote, with electronic circuitry off the patch. “Independent electrode” also includes electrodes that operate partially independently. Electrical wound parameters can be used to assess healing progress, as well as to tailor the signals applied to the wound in a closed-loop system that optimizes the healing process (rate, scarring, etc.). The system can also control and optimize non-electrical functions integrated into the patch, such as drug delivery and environmental control (oxygen, humidity and temperature).

While preferred embodiments of the present invention have been shown and described herein, it will be obvious to those skilled in the art that such embodiments are provided by way of example only. Numerous variations, changes, and substitutions will now occur to those skilled in the art without departing from the invention. Various alternatives to the embodiments of the invention described herein may be employed in practicing the invention. It is intended that the following claims define the scope of the invention and that methods and structures within the scope of these claims and their equivalents be covered thereby.

What is claimed is:

1. A wound-healing patch comprising:
a flexible substrate;
at least one wound electrode positioned on a portion of said flexible substrate adapted to be placed over wounded tissue; and
at least one return electrode positioned on said substrate remote from said wound-healing electrode, wherein said return electrode is positioned on a portion of said substrate adapted to be placed over healthy tissue.

2. A wound-healing patch according to claim 1 further comprising a voltage source connected between at least one of said wound electrodes and at least one of said return electrodes.

3. A wound-healing patch according to claim 1 further comprising a current source connected between at least one of said at least one wound electrode and at least one of said at least one return electrodes.

4. A wound-healing patch according to claim 1 further comprising at least one resistor connected to at least one of said at least one wound electrode.

5. A wound-healing patch according to claim 1 further comprising control electronics adapted to control the flow of current through said at least one wound electrode and said at least one return electrode.

6. A wound-healing patch according to claim 1 wherein one of said at least one wound-healing electrode or said at least one return electrode is electrically isolated from tissue when said patch is attached to said tissue.

7. A wound-healing patch according to claim 1 wherein both said at least one wound-healing electrode and said at least one return electrode are electrically isolated from tissue when said wound-healing patch is attached to tissue.

8. A wound-healing patch according to claim 7 wherein at least one said wound electrode or said at least one return electrode are isolated from said tissue by a distributed resistive element.

9. A wound-healing patch according to claim 1 wherein the collective area of said at least one return electrode is substantially larger than the collective area of said at least one wound electrode.

10. A wound-healing patch according to claim 1 wherein the collective area of said at least one return electrode is substantially smaller than the collective area of said at least one wound electrode.
